(19) United States
(12) Patent Application Publication
(10) Pub. No.: US 2003/0212112 A1
(43) Pub. Date: Nov. 13, 2003

(54) Method for administering a phosphodiesterase 4 inhibitor

(75) Inventors: Robert D. Murdoch, Harlow (GB); Theodore T. Torphy, Bryn Mawr, PA (US); Barry D. Zussman, Welwyn (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/429,666

(22) Filed: May 2, 2003

Related U.S. Application Data
(63) Continuation of application No. 10/111,957, filed on Apr. 29, 2002, now abandoned.
Application No. 10/111,957 is a 371 of international application No. PCT/US00/29453, filed on Oct. 26, 2000.
(60) Provisional application No. 60/162,477, filed on Oct. 29, 1999.
(60) Provisional application No. 60/162,641, filed on Nov. 1, 1999.
(60) Provisional application No. 60/179,817, filed on Feb. 2, 2000.

(51) Int. Cl. A61K 31/277; A61K 31/4439

(52) U.S. Cl. 514/340; 514/521

(57) ABSTRACT

This invention relates to a method for increasing the dose of a PDE4 inhibitor that can be administered at one time and be tolerated by the patient by reducing the absorption rate or the rate of rise in plasma concentration of the inhibitor.

### AREA OF THE INVENTION

This invention relates to an improved method for treating a patient with a drug, which inhibits the phosphodiesterase, 4 (PDE4) isozyme in a manner, which avoids side effects while increasing systemic exposure (e.g., area under the curve). This method involves decreasing the rate of rise of the drug in the plasma and/or delaying the onset of absorption of the drug. The result is that, at a given dose of drug the side effects which may occur with the drug at that plasma concentration from an immediate release formulation can be eliminated or substantially reduced in frequency of occurrence or severity, or the dose of the drug can be increased substantially while avoiding one or more if not all of the adverse side effects sometimes associated with it.

### BACKGROUND OF THE INVENTION

Cyclic nucleotide phosphodiesterases (PDEs) represent a family of enzymes that hydrolyze the ubiquitous intracellular second messengers, adenosine 3′,5′-monophosphate (cAMP) and guanosine 3′,5′-monophosphate (cGMP) to their corresponding inactive 5′-monophosphate metabolites. At least ten distinct classes of PDE isozymes are believed to exist, each possessing unique physical and kinetic characteristics and each representing a product of a different gene family. These are distinguished using Arabic numerals 1-10.

The enzyme targeted in this invention is the PDE4 isozyme in all its various forms and in the full domain of its distributions in all cells. It is a low Kₘ(cAMP Kₘ=1-5 μM) cAMP-selective enzyme that has little activity against cGMP (Km>100 μM).

Current PDE inhibitors used in treating inflammation and as bronchodilators, drugs like theophylline and pentoxyfyllin, inhibit PDE isozymes indiscriminately in all tissues. These compounds exhibit side effects, apparently because they non-selectively inhibit all PDE isozyme classes in all tissues. The targeted disease state may be effectively treated by such compounds, but unwanted secondary effects may be exhibited which, if they could be avoided or minimized, would increase the overall therapeutic effect of this approach to treating certain disease states.

A new approach toward improving the side effect profile of PDE inhibitors is to design a new generation of compounds that inhibit only a single PDE isozyme, i.e., the PDE isozyme that predominates in the tissue of cell of interest. The predominate cAMP PDE isozyme in immune and inflammatory cells is PDE4. It is also a major regulator of cAMP content in airway smooth muscle. Thus, selective inhibition of PdE4 elevates cAMP content in immune and inflammatory cells, as well as in airway smooth muscle. This leads to anti-inflammatory effects as well as bronchodilation. One or both of these therapeutic actions are useful in treating a variety of diseases, including, but not limited to asthma and COPD. PDE4 inhibitors, particularly PDE4-specific inhibitors are useful also in treating other diseases in the area of inflammation, (e.g., asthma, chronic obstructive pulmonary disease, inflammatory bowel disease, rheumatoid arthritis), affects related to tumor necrosis factor and to cognition impairment (e.g., multi-infarct dementia, cognitive dysfunction, or stroke).

Although in theory isozyme-selective PDE inhibitors should represent an improvement over non-selective inhibitors, the selective inhibitors tested to date are not devoid of side effects produced as an extension of inhibiting the isozyme of interest in an inappropriate or untargeted tissue, or because they may have cross-reactivity with other PDE isozymes. For example, clinical studies with the selective PDE4 inhibitor rolipram, which was being developed as an antidepressant, indicate it has psychotropic activity and produces gastrointestinal effects, e.g., pyrosis, nausea and emesis. Indications are that side effects of denbufylline, another PDE4 inhibitor targeted for the treatment of multi-infarct dementia, may include pyrosis, nausea and emesis as well. These side effects are thought to occur as a result of inhibiting PDE4 in specific areas of the central nervous system and the gastrointestinal system.

While to date no one has been able to identify a compound which is completely without unwanted side effects at all possible dosage levels, at least one compound has been identified that appears to be better tolerated than previous PDE4 inhibitors, namely cis-4-cyano-4-[3-(cyclopentyloxy)-4-methoxyphenyl]cyclohexane-1-carboxylic acid. Ariflo® is the registered trademark for this compound. And while Ariflo® appears to have an improved therapeutic ratio and can be administered orally to achieve an effective therapeutic effect in COPD at certain doses, it has been found that as plasma levels increase with increased levels of dosing using an immediate release oral tablet, undesirable side effects such as those attributed to CNS activity begin to be manifested.

To address this potential limitation, a major effort was initiated to identify methods whereby the dose of Ariflo®, and thus the plasma concentrations achieved, could be increased without causing concomitant side effects. The purpose of this work was to determine whether a greater therapeutic effect could be provided by increasing the systemic exposure or plasma levels of the compound without producing side effects. It has been found that increasing the dose level and consequently the plasma concentration of Ariflo® while avoiding or minimizing side effects can be achieved by either delaying the onset of absorption, decreasing the rate of rise of plasma levels of Ariflo®, or both. The effects were achieved using one possible iteration of this invention, a controlled or sustained release formulation. The controlled release formulations allowed for administering in a single dosage form several times the quantity that can otherwise be administered of a drug which inhibits PDE4 while achieving both initial therapeutically effective plasma levels and maintaining these plasma levels for an extended period of time. Accordingly, this invention provides a new method for administering a drug which inhibits PDE4 while avoiding or minimizing side effects by decreasing the rate of rise of the drug in the plasma or delaying the onset of absorption of the drug; the phenomena can be combined as well. This can also provide a means for increasing the dose of the drug substantially while avoiding or not increasing the adverse side effects that may be associated with it as compared with administering the same drug as an immediate release preparation or where it is immediately absorbed.

### SUMMARY OF THE INVENTION

In a first aspect this invention relates to a method for increasing the dose or systemic exposure of a drug which inhibits PDE4 over that administered in a treatment at a single point in time by at least about 2-fold and reducing the severity of or eliminating or avoiding the occurrence of one or more side effects, the method comprising formulating a controlled-release preparation comprising said drug and at least one pharmaceutically acceptable excipient capable of forming a controlled release formulation which delays appearance in the plasma of detectable amounts of said drug and wherein the resulting rate of rise in plasma concentration of said drug is at least about 10% less than that of an immediate release formulation containing the same amount of drug administered by the same route and administering said formulation to a patient.

In a second aspect this invention relates to a method for reducing the severity of or eliminating or avoiding the occurrence of one or more side effects of a drug which inhibits PDE4, the method comprising administering the drug in a formulation and/or in a manner which results in a reduction in the of rate of rise in plasma concentration of said drug by at least about 10% less than that of an immediate release formulation containing the same amount of drug administered by the same route.

In further aspect this invention relates to a method for reducing the severity of or eliminating or avoiding the occurrence of one or more side effects of a drug which inhibits PDE4, the method comprising administering the drug in a formulation and/or a manner which results in a delay in the appearance in the plasma of detectable amounts of said drug and results in a reduction of rate of rise in plasma concentration of said drug by at least about 10%, as compared with that of an immediate release formulation containing the same amount of drug administered by the same route.

In a further aspect this invention relates to a method for increasing the dose or systemic exposure of a drug which inhibits PDE4 administered in a treatment at a single point in time by at least about 2-fold and reducing the severity of or eliminating or avoiding the occurrence of one or more side effects, the method comprising administering a formulation containing the drug and at least one pharmaceutically acceptable excipient in a manner which results in a reduction of rate of rise in, plasma concentration of said drug by at least about 10% less than that of an immediate release formulation containing the same amount of drug administered by the same route.

In a further aspect, this invention relates to an improved process for administering a drug which inhibits PDE4 to patients suffering from or susceptible to a disease treatable by administering such a drug wherein the drug causes side effects related to inhibiting PDE4 when administered as an immediate release formulation, the improvement comprising formulating the drug as a formulation which delays the onset of absorption as measured by the appearance of said drug in the plasma and results in a reduction of rate of rise in plasma concentration of said drug by at least about 10% less than that of an immediate release formulation containing the same amount of drug administered by the same route.

In a yet further aspect this invention relates to an improved process for administering a drug which inhibits PDE4 to patients suffering from or susceptible to a disease treatable by a drug which inhibits PDE4 wherein the drug, when administered in as immediate release preparation, causes side effects related to the inhibition of PDE4, the improvement comprising formulating the drug as a preparation which delays the onset of absorption as measured by the appearance of detectable amounts of said inhibitor in the plasma and wherein the rate of rise in plasma concentration is reduced by at least about 10% as compared with that of an immediate release formulation containing the same amount of inhibitor administered by the same route.

In an additional aspect, this invention relates to an improved process for increasing the dose or systemic exposure of a drug which inhibits PDE4 that can be administered to a patient while reducing the severity of or eliminating or avoiding the occurrence of one or more side effects associated with administering an immediate release formulation containing the same or a lesser amount of said drug, the improvement comprising formulating the drug as a controlled release formulation which delays the onset of absorption as measured by the appearance of detectable amounts of said drug in the plasma results in a reduction of rate of rise in plasma concentration of said drug by at least about 10% compared with that of an immediate release formulation administered by the same route.

### DESCRIPTION OF THE FIGURES

FIG. 1 is a graph of plasma levels of Ariflo® for several doses given in the form of release tablets.

FIG. 2 is a bar chart of adverse events associated with the several different doses of Ariflo® administered as immediate release tablets.

FIG. 3 is a graph of the mean of steady state plasma concentration versus time profiles for two CR formulations and an IR formulation containing Ariflo®.

### DETAILED DESCRIPTION OF THE INVENTION

The improvement in therapy when using PDE4-inhibiting drugs has been found to lie in delaying the onset of absorption of the drug and/or reducing the rate of absorption of that inhibitor. Either or both approaches accomplish two things: 1) they permits one to increase the total amount of drug that is administered at a single time-point, resulting in increased plasma concentrations or systemic exposure; and 2) this higher dose avoids all or most of the side effects, or substantially reduces their occurrence and/or severity, as compared with an immediate release preparation that contains much less drug. Obviously one can also apply this invention to avoiding or reducing the occurrence of or the severity of side effects of a dose equal to that of an immediate release preparation as well.

As regards increasing the therapeutic index (more drug fewer side effects), one can increase by at least about 2-fold the amount of PDE4-inhibiting drug administered at a given time, as compared with an immediate release preparation. In fact it has been found that a 60 mg controlled release oral tablet has fewer side effects than those produced by a 20 mg immediate release oral tablet. It is has been demonstrated that it is possible to increase the one-time dose of a PDE inhibitor by 2- or 3-fold, and it is possible to increase it 4- or 5-fold if the excipients are selectively chosen and optimized, the route of administration is optimized, the physical form of the drug is taken into consideration, or a combination of I or more of these and other possible factors are taken into account.

This invention covers administering formulations, which contain a drug, which inhibits the PDE4 isozyme and which cause some side effects when administered as an immediate release preparation. A preferred sub-group such drugs are those which specifically inhibit PDE4. A more preferred group are those drugs that have an IC₅₀ ratio (high/low binding) of about 0.1 or greater as further described in U.S. Pat. No. 5,998,428 and its counter-part PCT application serial number WO95/00139 published Jan. 05, 1995. This U.S. patent is incorporated herein in full by reference as if fully set forth herein. Without limiting the practice of this invention, other, exemplary, PDE4 inhibitors that may be included in these formulations include the following inhibitors:

Some representative compounds, which are useful in this invention, are set out in U.S. Pat. No. 5,552,438 issued Sep. 03, 1996. This patent and the compounds it discloses are incorporated herein in full by reference. The compound of particular interest, which is disclosed in U.S. Pat. No. 5,552,438, is cis-4-cyano-4-[3-(cyclopentyloxy)-4-methoxyphenyl]cyclohexane-1-carboxylic acid and its salts, esters, pro-drugs or physical forms.

AWD-12-281 from Astra (Hofgen, N. et al. 15th EFMC Int Symp Med Chem (Sep. 6-10, 1998 Edinburgh), Abst P.98); a 9-benzyladenine derivative nominated NCS-613 (INSERM); D4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (Sep. 19-23, 1998 Geneva) 1998, 12(Suppl. 28): Abst P2393); roflumilast (CAS reference No 162401-32-3) and a pthalazinone (WO 9947505) from Byk-Gulden; or a compound identified as T-440 (Tanabe Seiyaku; Fuji, K. et al. J Pharmacol Exp Ther, 1998, 284(1): 162). Any one or all of these compounds may or could benefit from the process described herein.

The achievements of the improvement provided by this invention is that of modulating the release, absorption, administration or conversion of an inhibitor in a manner which either reduces the rate of rise of the active moiety of the drug as measured by plasma concentrations and/or delays the absorption of the drug or a precursor of the active moiety of the drug. The standard for comparison against which to measure the reduced rate of rise and/or delayed onset of absorption is that of an immediate release formulation administered by the same route, at the same time and under the same conditions using the same moiety.

Any number of means can achieve these goals. Some examples are: controlled release preparations; coated beadlet technologies; capsule micropump technology; ramp infusion; suspensions or vehicles which have a depot affect such as the administration of thixotropic preparations. Examples and methods describing and teaching how to make formulations of this type are available in texts like Remington's Pharmaceutical Sciences, 18th Edition, Mack Publishing Co. Easton, Pa., U.S.A. 18042 or later additions or Drugs and Pharmaceutical Sciences, v 29 and “Controlled Drug Delivery: Fundamentals and Applications, Second Edition,” Edited by Joseph R. Robinson and Vincent H. Lee, Published by Marcel Dekker Inc.

One can also manipulate the physical form of the drug to achieve delayed onset of absorption or a decreased rate of rise by administering the drug as a polymorph, solvate, hydrate or the like; or using pro-drugs, or salts, for example.

Route of administration is not a critical factor. This invention has application irrespective of the route of administration. It will have greatest application to formulations administered orally, bucally, nasally, by inhalation, by suppository, by IV injection, sub-cutaneously or by intramuscular injection. It can also be applied to topical preparations such as salves, ointments, and dermal patch technologies, as well as IP injections or ocular preparations.

At least a 10 minute delay in onset of increasing plasma concentrations is a preferred practice, although a delay of somewhere between 10 and 45 minutes, say 30 minutes, or greater (1 hour or more) also useful and practicable. This timing of delay applies to all forms of practicing this invention, not just to the preferred preparations such as the controlled release tablets. It can be measured by reference to the onset of absorption as measured against an immediate release (IR) tablet, although an IR formulation is but one possible standard.

A 10% reduction in rate of rise of plasma concentration of drug is a threshold for eliminating or reducing their occurrence or severity side effects with a given dose of drug or for increasing the amount of drug that is administered, either at a single point in time or when a titration or infusion technique is used. Such reduction is determined by comparison to an immediate release preparation administered by the same route. For example, if the oral route of administration is chosen as the approach to be taken, then an immediate release tablet or capsule is the standard against which to measure the 10% reduction in rate of rise. A greater reduction in rate of rise is also within the scope of this invention, i.e., 10-25%, including a 15-20% or 20-25%, or greater percentage.

For the purposes of this invention, it is preferred to manufacture a product which contains between about 1 mg to 200 mg, more preferably 5 to 100 mg, most preferably between 5 or 10 to 60 mg of the active ingredient. Additional preferred dosage amounts within these ranges are 10, 15, 20, 30, 40, 50, 60, 70, 80 or 90 mg per preparation.

A preferred methodology for reducing the rate of rise and/or delaying onset of absorption is the controlled release technologies. This involves formulating drug with excipients, which modulate and extend the period over which the active ingredient is released from the carrier. Herein the term “controlled release” (CR) is used. This type of formula is sometimes described as a sustained release formulation or a non-immediate-release delivery system. “Controlled release” is intended to cover any formulation which can be characterized as having a release profile wherein a portion of its drug load is released over time, either episodically or continuously over time. It also includes preparations where the initial release of drug is delayed as a result of an external barrier or coating which is selectively soluble in the environment where the formulation is placed, or a preparation where the coating does not break down in the environment into which the formulation is initially introduced, but then migrates to another environment in which the external coating is soluble or is broken down, after which the drug is released over time. By way of further illustration and explanation, these delivery systems can be characterized as: i) delayed release, ii) controlled or prolonged release, iii) site-specific release, or iv) receptor release. A more detailed explanation-of these different systems is available in the likes of Remington's Pharmaceutical Sciences, 18th Edition, Mack Publishing Co. Easton, Pa., U.S.A. 18042 or later additions or Drugs and Pharmaceutical Sciences, v 29: “Controlled Drug Delivery: Fundamentals and Applications, Second Edition,” Edited by Joseph R. Robinson and Vincent H. Lee, Published by Marcel Dekker Inc.

The preferred forms of this invention are the oral delayed release formulations. These systems may be dissolution-dependent as illustrated by encapsulated dissolution products or matrix dissolution products. Or they may be formulated using osmotic systems or ion exchange resins. The most preferred approach is to provide an oral controlled release product based on matrix dissolution technology.

A delay in onset of absorption and a reduced rate of absorption were correlated with reduced side effects when a known PDE4 inhibitor was administered to patients in an oral CR preparation containing 3 times the amount of drug as that of an immediate release tablet which was not well tolerated. It was also observed that the CR tablet resulted in a reduced rate of absorption, reflected in a reduced rate of rise in plasma concentrations but gave a Cₘₐₓ several hours later which exceeded the Cₘₐₓ associated with adverse side effects in an immediate release tablet. That is, when an immediate release tablet is given to a patient, the Cₘₐₓ and side effects correlate strongly. This correlation was not observed with the CR formulation even though it resulted in a higher Cₘₐₓ. It is not clear whether the results seen with a CR preparation are dependent on or related to one or both of these observations, or to another factor or combination of factors, such as Tₘₐₓ, ka, Tlag (absorption lag-time), or some other factor or phenomenon. Regardless of what the underlying mechanism is, the result is that it is now possible to significantly increase the amount of PDE4 inhibitor administered at a particular time point and avoid the tolerability challenges associated with PDE4 inhibitors in certain segments of the population. And get to plasma levels, which provide a therapeutic effect in a target disease.

Controlled release preparations exemplified in this invention can be prepared by selecting excipients from any number or type of materials which provide the requisite controlled release profile needed to avoid side effects while allowing for a significant increase in the amount of drug contained in the formulation, as compared with an immediate release preparation. Without intending to be limited, a preferred approach is to use a matrix dissolution technology based on acrylic acid polymers. Carbomer is the non-proprietary name for these materials. They are high molecular weight polymers prepared by cross-linking acrylic acids with the likes of allylsucrose or allyl ethers of pentaerythritol. Such polymers also go by the name acritamer or carbopol. The chemical name and CAS registry number for the class is carboxypolymethylene [54182-57-9]. Exemplary carbomers are carbomer 910 [91315-32-1], carbomer 934 [9007-16-3], carbomer 934P [9003-01-4] and carbomer 940 [76050-42-5]. These polymers contain between 56-68% of carboxylic acid groups, calculated on a dry basis. A blend of two or more carbomers of differing molecular weight can be used to modify and manipulate the release rate. Examples are given below. In addition, the preferred formula may contain a binding agent, tillers, lubricants, and the like.

The preferred excipients for affecting release rate are carbomers, particularly a combination of two or more different carbomers. Especially preferred are those carbomers known as Carbopols and are manufactured by BF Goodrich. Preferred carbomers are: Carbomer 934P (Carbopol 974P) and Carbomer 941P (Carbopol 971P).

A preferred formulation will have between about 1-25% by weight of drug, preferably an amount between 3-20% and optionally an amount between about 5 and 15%. Other specific amounts are set out in the Examples below. In regards to the carbomers, one or more may be used to realize the controlled release effect. It is preferred to use two carbomers in a given formulation. When a preferred formulation containing the acid set out above is prepared, one or both of two carbomers is used in a range between 0-9% each. These percentages are weight/weight percentages. Further specific preferred percentages of carbomers are given in the Examples set out below.

The following examples are provided to illustrate how to make and use the invention. They are not in any way intended to limit the scope of the invention in any manner or to any degree. Please refer to the claims for what is reserved to the inventors hereunder.

### EXAMPLE 1

Immediate Release Tablets

Immediate release tablets used in patient studies investigating the tolerability of Ariflo® in humans were prepared by standard means and contained the ingredients set out in Table 1.

TABLE 1
Immediate Release Tablets

| | Quantity | Quantity | Quantity |
|---|---|---|---|
| Ingredients | (mg/tablet) | (mg/tablet) | (mg/tablet) |
| Ariflo ® | 5.0 | 10.0 | 15.0 |
| Lactose Monohydrate | 113.0 | 108 | 103 |
| Microcrystalline Cellulose | 70.0 | 70.0 | 70.0 |
| Sodium Starch Glycolate | 10.0 | 10.0 | 10.0 |
| Magnesium Stearate | 2.0 | 2.0 | 2.0 |
| Opadry White OY-S-9603 | 5.0 | 5.0 | 5.0 |
| Total Tablet Weight (mg) | 205.0 | 205 | 205 |

### EXAMPLE 2

Treatment with IR Formulations and Occurrence of Adverse Events

A double blind, placebo controlled, parallel group study was carried out to determine the safety, tolerability and pharmacokinetics of Ariflo® in healthy male subjects.

Ninety-four healthy male, non-smoking subjects aged 18-45 years with no known intolerance to theophylline or theophylline derivatives were selected. Subjects received a single dose of 2, 4, 7, 10, 15 or 20 mg/placebo on day 1, 48 hr later subjects received the same dose as on day 1, bid for the next 6.5 days. Each dose was a capsule containing the excipients and manufactured as illustrated in Example 1. The instructions were to take each capsule orally with water 2 hours before food. The period between the single and repeat dose phases was supplemented by placebo to maintain compliance with the dosing regimen.

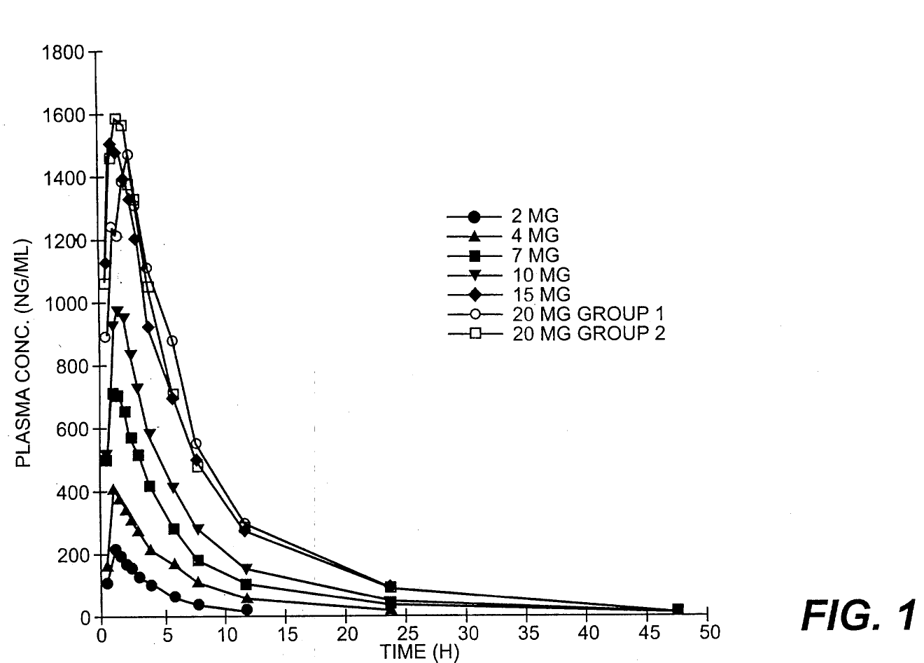

Plasma levels for each dose at each timepoint where blood was drawn are given in FIG. 1.

Adverse event forms were completed predose and thereafter at 12 hourly intervals during the study and again at follow-up. Spontaneously reported adverse events were also recorded.

A total of 236 post dose adverse events were reported for 68 of the 94 subjects exposed to medication. Only 12 of these were classified as severe and occurred in 9 subjects. These included nausea, headache, vomiting, dyspepsia, back pain and dizziness. The most frequent treatment emergent adverse events by dose are shown in Table 2.

TABLE 2
Adverse Events: IR Formulation

| Adverse | | | | | | | |
|---|---|---|---|---|---|---|---|
| Event | Placebo | 20 mg* | 15 mg | 10 mg | 7 mg | 4 mg* | 2 mg |
| Headache | 6 | 6 | 5 | 3 | 6 | 8 | 3 |
| Dizziness | 5 | 9 | 1 | 1 | 1 | 2 | 2 |
| Nausea | 3 | 12 | 6 | 3 | 0 | 5 | 1 |
| Vomiting | 0 | 7 | 1 | 0 | 1 | 2 | 1 |
| Abdom- | 0 | 5 | 0 | 0 | 2 | 0 | 0 |
| inal pain | | | | | | | |
| Rigors | 0 | 3 | 0 | 0 | 0 | 0 | 0 |
| ALT | 3 | 0 | 2 | 4 | 0 | 1 | 0 |
| elevation | | | | | | | |
| AST | 1 | 0 | 0 | 2 | 0 | 0 | 0 |
| elevation | | | | | | | |
| Subjects | 23 | 18# | 9 | 10 | 9 | 16 | 9 |
| exposed | | | | | | | |

*Includes subjects withdrawn as a result of dosing error.
#No subject in the two 20 mg cohorts completed the study.

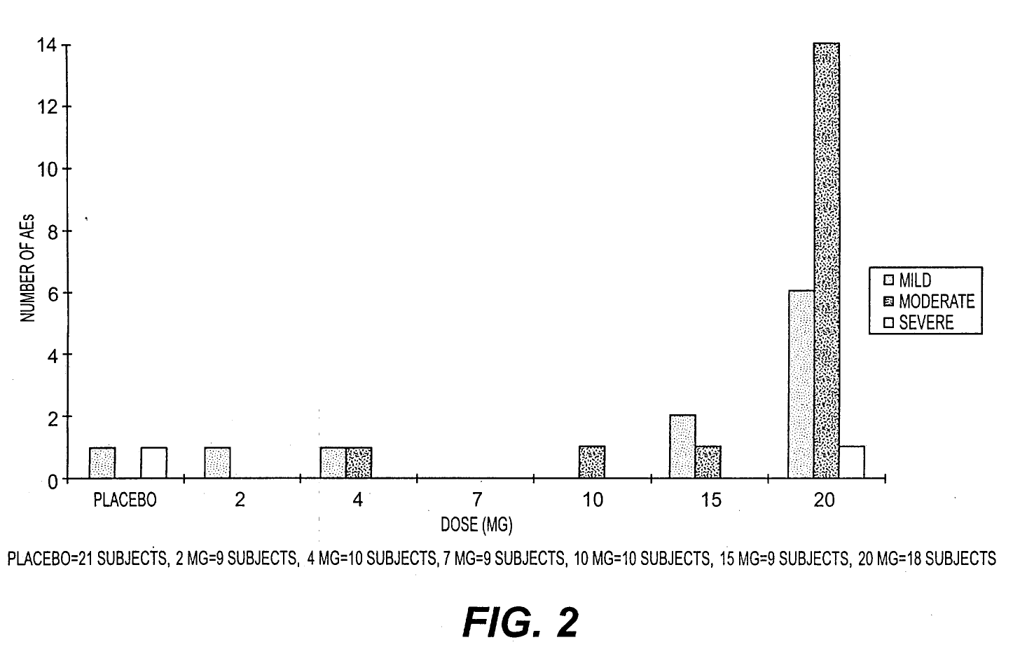

FIG. 2 summarizes the adverse event data in Table 2 in bar-graph form.

The incidence of adverse events up to and including 10 mg was indistinguishable from placebo. At 15 mg, nausea with a single episode of vomiting was seen. These events occurred only on day 1 or the first day of the repeat dose phase and there were no protracted episodes of nausea during the week of dosing. Nausea was also reported on placebo. At 20mg, the adverse event profile on day I and on day I of the repeat dose phase indicated that the compound was not well tolerated and the study was terminated. The characteristics that differentiated the 15 mg group from the 20 mg group was not only an increase in the incidence of nausea but the appearance of vomiting, dizziness, abdominal pain and rigors in a significant number of subjects.

### EXAMPLE 3

Dose Titration Study

A double-blind, placebo-controlled, two-part dose-escalation study was carried out to determine the tolerability and pharmacokinetics of increasing doses of Ariflo® (up to 60 mg daily, in divided doses) in healthy male volunteers in the fed state.

The study was carried out by administering to subjects the immediate release tablet prepared as per Example 1 and containing 10 mg, 15 mg, 20 mg, 25 mg and 30 mg (or placebo) b.i.d. after breakfast and dinner, according to the following dose-rising schedule:

| | |
|---|---|
| Days 1-3 | 10 mg SB-207499 b.i.d. or placebo b.i.d. |
| Days 4-6 | 15 mg SB-207499 b.i.d. or placebo b.i.d. |
| Days 7-9 | 20 mg SB-207499 b.i.d. or placebo b.i.d. |
| Days 10-12 | 25 mg SB-207499 b.i.d. or placebo b.i.d. |
| Days 13-15 | 30 mg SB-207499 b.i.d. or placebo b.i.d. |

This study showed that by utilising a dose-escalation regimen (10, 15, 20, 25 and 30 mg bid with food for 3 days at each dose level with the IR formulation of Example 1, a well-tolerated daily dose of 30 mg bid was achieved. The mean plasma blood levels for Ariflo® are set out in the graph of FIG. 1 as a solid line intersected by solid dots.

### EXAMPLE 4

Controlled Release Formulation—Different Drug Loads

A set of controlled release tablets was prepared containing 5 different drug concentrations. Tablets were prepared as follows using the excipients set out in Table 3:

Blending

The blends were made up in using the components listed in Table 3. All excipient except and drug, except for magnesium stearate, were placed in a blender and mixed. The magnesium stearate was then added and blended for an additional 3 minutes. During the blending process, excipients and drug were mixed, passed through a screen and then mixed again.

Compression

Approximately 350 mg of each mix was compressed into tablets. A target tablet strength of 10 kp was used.

TABLE 3
Composition of Tablets

| Component | Weight of Component in Milligrams | | | | |
|---|---|---|---|---|---|
| Drug (Ariflo ®) | 20 | 30 | 40 | 50 | 60 |
| Dibasic Ca Phosphate | 259 | 249 | 239 | 229 | 219 |
| Carbomer 934P | 9 | 9 | 9 | 9 | 9 |
| Carbomer 941P | 9 | 9 | 9 | 9 | 9 |
| Magnesium Stearate | 3 | 3 | 3 | 3 | 3 |
| Opadry White | 7.5 | 7.5 | 7.5 | 7.5 | 7.5 |
| OY-S-9603 | | | | | |
| Purified Water | q.s. | q.s. | q.s. | q.s. | q.s. |
| Total Tablet Wt. (mg) | 307.5 | 307.5 | 307.5 | 307.5 | 307.5 |

Opadry White was suspended in the purified water and this suspension was used to coat the tablets; the water evaporated after the tablets were coated and did not form any part of the final product.

A typical in vitro dissolution profile for these tablets is given in Table 4.

TABLE 4
Dissolution Profile

| Time (hrs) | % Released |
|---|---|
| 0 | 0 |
| 1 | 9 |
| 3 | 38 |
| 5 | 63 |
| 8 | 83 |
| 12 | 95 |

### EXAMPLE 5

Controlled Release Formulation

Three sets of controlled release formulations were prepared using the blending and compression techniques described in Example 4. One set was formulated to give a fast release rate. The second and third-formulations were designed to give a medium and slow release rate. Specific details for each set of tablets is given in Table 5.

TABLE 5
Table Ingredients

| | Fast | Medium | Slow |
|---|---|---|---|
| | % w/w | % w/w | % w/w |
| Drug (Ariflo) | 3.3 | 3.3 | 3.3 |
| Dibasic Calcium Phosphate | 88.0 | 88.5 | 88.5 |
| (anhydrous) | | | |
| Carbomer 934P | 5.4 | 3.3 | 0.0 |
| Carbomer 941P | 0.0 | 1.6 | 4.9 |
| Magnesium Stearate | 1.0 | 1.0 | 1.0 |
| Opadry White OY-S-9603 | 2.4 | 2.4 | 2.4 |
| Purified water | q.s. | q.s. | q.s. |

Opadry White was suspended in the purified water and that suspension was used to coat the tablets; the water evaporated after the tablets were coated and did not form any part of the final product.

These formulations gave in-vitro dissolution data (% released) as per Table 6.

TABLE 6
Release Profile Over Time

| Time (hrs) | Fast | Medium | Slow |
|---|---|---|---|
| 0 | 0 | 0 | 0 |
| 1 | 21 | 15.3 | 8 |
| 2 | 41 | 28 | 15 |
| 3 | 68 | 43 | 22 |
| 5 | 97 | 68 | 36 |
| 8 | 100 | 87 | 51 |
| 12 | 100 | 98 | 69 |
| 18 | — | — | 90 |
| 24 | — | — | 101 |

### EXAMPLE 6

Controlled Release Formulations

Controlled release tablets were prepared containing five different drug loads. Ingredients and the amount of each ingredient per drug load are set out in Table 7. Tablets were prepared as described in Example 3.

TABLE 7
Controlled Release Formulation Preparations

| | 20 | 30 | 40 | 50 | 60 |
|---|---|---|---|---|---|
| | mg | mg | mg | mg | mg |
| Tablet Core Components | | | | | |
| Ariflo ® | 20.0 | 30.0 | 40.0 | 50.0 | 60.0 |
| Dibasic Calcium Phosphate, | 259.0 | 249.0 | 239.0 | 415.0 | 498.0 |
| Anhydrous | | | | | |
| Carbomer 934P (Carbopol 947P) | 9.0 | 9.0 | 9.0 | 15.0 | 18.0 |
| Carbomer 941 (Carbopol 971P) | 9.0 | 9.0 | 9.0 | 15.0 | 18.0 |
| Magnesium Stearate | 3.0 | 3.0 | 3.0 | 5.0 | 6.0 |
| Tablet Core Weight—Total | 300.0 | 300.0 | 300.0 | 500.0 | 600.0 |
| Coating Component | | | | | |
| White Opadry (OY-S-9603) | 7.5 | 7.5 | 7.5 | 12.5 | 15.0 |
| Tablet Weight—Total | 307.5 | 307.5 | 307.5 | 512.5 | 615.0 |

### EXAMPLE 7

Comparison of an IR Tablet and a CR Tablet

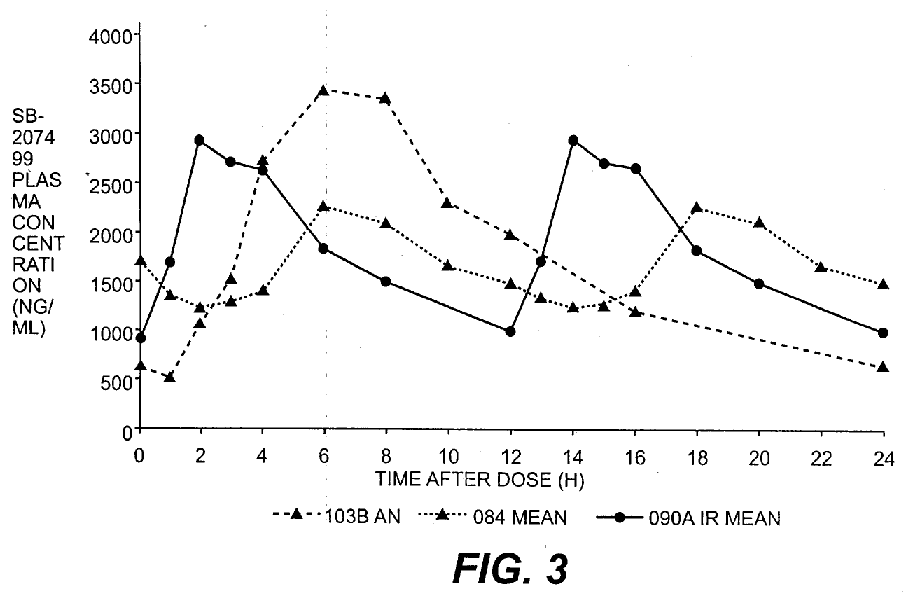

In a first study, Ariflo® was administered as conventional immediate-release (IR) tablets at a range of single and multiple (bid) doses. A 15 mg tablet (dosed singly and repeatedly) was reasonably well tolerated, but a single 20 mg dose was so poorly tolerated that multiple dosing was not attempted. It was observed that this tolerability problem related in some way to plasma concentrations since the adverse experiences (mostly gastrointestinal) were observed at or close to Cₘₐₓ. The Cₘₐₓ for the 15 mg IR tablet occurred at about 2 hours. While a 15 mg dose of Ariflo® in IR tablet was very effective in treating COPD, certain types of asthma did not respond optimally to that dose delivered as an immediate release tablet. To achieve daily doses higher than the 15 mg bid alternative dosing strategies were developed, ones which could increase total daily exposure (area under the curve) while possibly overcoming the problem of what appeared to be concentration-related GI adverse experiences at Cₘₐₓ with the IR tablet. A CR formulation was investigated based on an observation in one study that a 30 mg bid regimen of an IR tablet could achieve the goals of tolerability and an increased AUC by an up-titration regimen in which the dose was increased progressively from 10 to 30 mg bid (FIG. 3: solid line with solid circles). This was new information in regards to PDE4 inhibitors, i.e., that slowly increasing the dose levels avoided most or all of the tolerability challenges and at the same time allowed the dosage to be increased. CR formulations were then developed to see if the CR characteristics protected patients from the rapid concentration rise and high concentration peak seen with the IR tablet, and associated with intolerable adverse experiences.

Candidate CR formulations with a range of dissolution profiles were compared; the formulation identified as “medium” in Table 5 above was used. Studies in humans showed that repeat-dose regimens of 60 mg uid or 30 mg bid were both well tolerated and the AUC was increased (FIGS. 1). It was not possible to determine the extent to which the delay in onset of absorption (lag time), or reduction in rate of rise contributed to the better tolerability of these two CR formulations. Nor did this work negate the possibility that one or more other pharmacokinetic factors (e.g. Tmax, ka, etc.) might contribute to the enhanced tolerability observed. Not withstanding the mechanism(s) of action remains obscure, the results are clear: AUC is significantly increased and side effects are greatly minimized or not present at all. Also, total bioavailability (AUC) was not significantly altered by formulating Ariflo® in the CR tablet described in Example 4. Table 8 sets out the results of these studies, which used as the IR formulations those, described in Example 1.

TABLE 8
Steady-state PK parameters (CR and IR, 60 and 30 mg/day)

| | Controlled-release* | | Immediate-release* | | |
|---|---|---|---|---|---|
| | 2 × 30 mg | 30 mg | 30 mg | 15 mg | 15 mg |
| | UID | BID | BID | BID | BID |
| Parameter | (n = 7) | (n = 8) | (n = 8) | (n = 8) | (n = 15) |
| Daily dose | 60 mg | 60 mg | 60 mg | 30 mg | 30 mg |
| Cmax | 4.13 | 2.43 | 3.05 | 1.34 | 1.70 |
| [ug/mL] | (25%) | (36%) | (18%) | (24%) | (28%) |
| | 2.92-5.35 | 1.09-3.75 | 2.37-3.96 | 1.02-1.79 | 0.98-2.48 |
| AUC(0-24) | 40.5 | 39.9 | 43.0 | 18.4 | 20.0 |
| [ug.h/mL] | (19%) | (34%) | (26%) | (30%) | (35%) |
| | 30.3-50.4 | 18.9-60.4 | 31.5-61.2 | 14.2-27.7 | 8.93-32.2 |
| Cpredose | 0.62 | 1.58 | 0.96 | 0.40 | 0.47 |
| (am.) | (34%) | (31%) | (44%) | (49%) | (48%) |
| [ug/mL] | 0.29-0.82 | 1.44-2.47 | 0.60-1.71 | 0.19-0.74 | 0.11-0.92 |
| Peak-trough | 9.6 | 2.3 | 3.5 | 3.9 | 4.3 |
| ratio | (42%) | (29%) | (31%) | (42%) | (48%) |
| [Cmax/ | 5.1-14.8 | 1.6-3.2 | 2.0-5.4 | 2.4-7.1 | 2.2-10.8 |
| Cmin] | | | | | |
| Tmax | 6.0 | 6.0 | 2.0 | 2.5 | 3.0 |
| [h] | 4.0-8.0 | 2.0-8.0 | 1.0-4.0 | 2.0-6.0 | 1.5-4.0 |

Pharmacokinetic (PK) data are presented as mean, between-subject coefficient of variation, and range (median and range of Tₘₐₓ).
*Administered with a meal

FIG. 3 graphs the concentration in ng/ml of Ariflo in plasma at selected time-points over 24 hours. The solid line intersected with dots represents data from the dose titration study of Example 3. The dotted line interrupted by solid triangles reflects plasma levels observed in man after administering a ³0mg controlled release formulation of Ariflo® second column under “Controlled Release” in Table 8 (tablet: Table 5: medium release rate). Lastly, the dashed line interrupted by solid triangles reflects plasma levels observed in man after administering a one-time dose of two 30 mg CR tablets; first column under “Controlled Release” in Table 8 (tablet: Table 5: medium release rate).

### EXAMPLE 8

Effect of Food

Studies were designed to examine the effect of food and antacids on Ariflo bioavailability and rate of absorption. In a first study subjects (n=28) received a single 15 mg Ariflo immediate release tablet prepared as per Example 7 on two occasions; fasted and after an US Food and Drug Administration high-fat breakfast. Table 9 gives the details of the high-fat breakfast

TABLE 9

2 slices of toast; 20 g butter; 50 g jam;
2 slices of bacon; 2 fried eggs (or 80 ml liquid egg);
1 portion of fried potatoes of about 125 g;
20 g butter added to bacon, eggs, potatoes;
20 g cream cheese with 60% fat (e.g. Philadelphia)
or 200 ml regular/full-cream milk;
caffeine-free coffee or fruit tea, sugar.

Treatments were randomized and doses were administered at weekly intervals. Blood samples were drawn up to 48 h post-dose, and plasma concentrations of Ariflo (measured by LC/MS/MS) were subjected to standard pharmacokinetic (PK) analysis.

The high-fat meal reduced the rate of absorption (Tmax 2 hours, Cmax 40%) but had no effect on bioavailability (AUC unchanged), as compared with the self-selected meal.

What is claimed is:

1. A method for increasing the dose or systemic exposure of a drug which inhibits PDE4 over that administered in a treatment at a single point in time by at least about 2-fold and reducing the severity of or eliminating or avoiding the occurrence of one or more side effects, the method comprising formulating a controlled-release preparation comprising said drug and at least one pharmaceutically acceptable excipient capable of forming a controlled release formulation which delays appearance in the plasma of detectable amounts of said drug and wherein the resulting rate of rise in plasma concentration of said drug is at least about 10% less than that of an immediate release formulation containing the same amount of drug administered by the same route and administering said formulation to a patient.

2. A method for reducing the severity of or eliminating or avoiding the occurrence of one or more side effects of a drug which inhibits PDE4, the method comprising administering the drug in a formulation and/or in a manner which results in a reduction in the of rate of rise in plasma concentration of said drug by at least about 10% less than that of an immediate release formulation containing the same amount of drug administered by the same route.

3. A method for reducing the severity of or eliminating or avoiding the occurrence of one or more side effects of a drug which inhibits PDE4, the method comprising administering the drug in a formulation and/or in a manner which results in a delay in the appearance in the plasma of detectable amounts of said drug and results in a reduction of rate of rise in plasma concentration of said drug by at least about 10%, as compared with than that of an immediate release formulation containing the same amount of drug administered by the same route.

4. The method of any one of claims 1-3 wherein the patient suffers from asthma.

5. The method of any one of claim 1-3 wherein the patient has chronic obstructive pulmonary disease.

6. The method of any one of claims 1-3 wherein the formulation comprises a drug in an amount of about 1-25% percent by weight, about 0-10% percent of carbopol 971P by weight, 0-10% percent of carbopol 974P by weight, and additional pharmaceutically acceptable excipients to make 100 percent by weight.

7. The method of any one of claims 1-6 wherein the drug is cis-4-cyano-4-[3-(cyclopentyloxy)-4-methoxyphenyl]cyclohexane-1-carboxylic acid or a salt, ester, pro-drug or a physical form thereof inan amount between 10 and 60 mg.

8. The method of any one of claim 1-6 wherein the drug is roflumilast.
